(19) United States
(12) Patent Application Publication
(10) Pub. No.: US 2026/0082195 A1
(43) Pub. Date: Mar. 19, 2026

(54) Methods and Systems of Optimizing Emergency Vehicle to Healthcare Facility Transport and Communications

(71) Applicant: T-Mobile Innovations LLC, Overland Park, KS (US)

(72) Inventors: Mark HOLLAND, Reston, VA (US); Joao TEIXEIRA, Overland Park, KS (US)

(21) Appl. No.: 18/889,310

(22) Filed: Sep. 18, 2024

(51) Int. Cl. H04W 4/90; G16H 10/60; G16H 40/63

(52) U.S. Cl. CPC H04W 4/90; G16H 10/60; G16H 40/63

(57) ABSTRACT

An emergency vehicle system is configured to obtain patient data associated with a patient based on identification data of the patient, obtain medical device data from a plurality of different medical devices deployed in the emergency vehicle, determine current patient condition data indicating a current condition of the patient based on at least one of the medical device data collected from the different medical devices or input received at the emergency vehicle system, identify using a predictive model in the communications network, a healthcare facility of a plurality of healthcare facilities for treatment of the patient based on the patient data, facility data, and route traffic data, and instruct a network element to transmit first medical device data from a first medical device along a network path in the communication network along to the healthcare facility based on a network profile associated with the first medical device data.

### CROSS-REFERENCE TO RELATED APPLICATIONS

None.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

When an emergency vehicle (e.g., an ambulance, helicopter, etc.) arrives at an emergency scene, medical personnel (e.g., a paramedic, emergency medical technician, first responder, etc.) may conduct a scene safety assessment and then quickly move to evaluate a condition of every individual at the scene. The medical personnel may first perform a primary assessment to identify any life-threatening injuries or medical emergencies before attempting to stabilize the patient. Once stabilized, the patient is carefully moved and secured into the ambulance for transport, and vital signs are continuously monitored as the ambulance navigates to an appropriate healthcare facility.

### SUMMARY

In an embodiment, a method implemented in a communication network to optimize emergency vehicle to healthcare facility transportation and communications is disclosed. The method comprises obtaining, by a vehicle application implemented by an emergency vehicle system in the communication network, patient data associated with a patient based on identification data of the patient, in which the patient data comprises historical patient data describing at least one of a diagnosis history of the patient, a treatment history of the patient, physician data indicating contact information of one or more current physicians of the patient, or allergy data identifying one or more allergies of the patient, obtaining, by the vehicle application, medical device data from a plurality of different medical devices deployed in the emergency vehicle, and obtaining, by the vehicle application, current patient condition data indicating a current condition of the patient based on at least one of the medical device data collected from the different medical devices or input received at the emergency vehicle system. The method further comprises identifying, by the vehicle application, using a predictive model in the communications network, a healthcare facility for treatment of the patient based on the patient data, the current patient condition data, facility data, and route traffic data, in which the facility data comprises at least one of a location data indicating a location of the healthcare facility or facility resource data describing an available capacity of resources at the healthcare facility, and the route traffic data indicates at least one of real-time road conditions, traffic congestions, or potential obstacles encountered along a route to the healthcare facility, generating, by the vehicle application, using the predictive model, a predicted treatment plan for the patient to be performed on the patient while the emergency vehicle is in transit to the healthcare facility, and instructing, by the vehicle application, a network element to transmit first medical device data from a first medical device along a network path in the communication network to the healthcare facility based on a network profile associated with the first medical device data.

In another embodiment, a method implemented in a communication network to optimize emergency vehicle to hospital transportation and communications is disclosed. The method comprises obtaining, by a vehicle application implemented by an emergency vehicle system in the communication network, patient data associated with a patient based on identification data of the patient, in which the patient data comprises historical patient data describing at least one of a diagnosis history of the patient, a treatment history of the patient, physician data indicating contact information of one or more current physicians of the patient, or allergy data identifying one or more allergies of the patient, obtaining, by the vehicle application, medical device data from a plurality of different medical devices deployed in the emergency vehicle, and identifying, by the vehicle application, using a predictive model in the communications network, a healthcare facility of a plurality of healthcare facilities for treatment of the patient based on the patient data, facility data, and route traffic data. The method further comprises determining, by the vehicle application, a first network profile for first medical device data received from a first medical device of the different medical devices deployed in the emergency vehicle based on a first policy associated with attributes of at least one of the first medical device or the first medical device data, and determining, by the vehicle application, a second network profile for second medical device data received from a second medical device of the different medical devices deployed in the emergency vehicle based on a second policy associated with attributes of at least one of the second medical device or the second medical device data. The method further comprises determining, by the vehicle application, a first network path in the communication network along which to route the first medical device data to the healthcare facility based on the first network profile and a second network path in the communication network along which to route the second medical device data based on the second network profile, and instructing, by the vehicle application, a network element in the emergency vehicle to forward the first medical device data to the healthcare facility along the first network path and the second medical device data along the second network path.

In yet another embodiment, an emergency vehicle system of an emergency vehicle is disclosed. The emergency vehicle system of an emergency vehicle comprises at least one processor, at least one memory coupled to the processor, and a vehicle application, stored in the at least one memory. The vehicle application, when executed by the at least one processor, causes the vehicle application to be configured to obtain patient data associated with a patient based on identification data of the patient, in which the patient data comprises historical patient data describing at least one of a diagnosis history of the patient, a treatment history of the patient, physician data indicating contact information of one or more current physicians of the patient, or allergy data identifying one or more allergies of the patient, obtain medical device data from a plurality of different medical devices deployed in the emergency vehicle, determine, by the vehicle application, current patient condition data indicating a current condition of the patient based on at least one of the medical device data collected from the different medical devices or input received at the emergency vehicle system, identify, by the vehicle application, using a predictive model in the communications network, a healthcare facility of a plurality of healthcare facilities for treatment of the patient based on the patient data, facility data, and route traffic data, and instruct, by the vehicle application, a network element to transmit first medical device data from a first medical device along a network path in the communication network along to the healthcare facility based on a network profile associated with the first medical device data.

These and other features will be more clearly understood from the following detailed description taken in conjunction with the accompanying drawings and claims.

### BRIEF DESCRIPTION OF THE DRAWINGS

For a more complete understanding of the present disclosure, reference is now made to the following brief description, taken in connection with the accompanying drawings and detailed description, wherein like reference numerals represent like parts.

FIG. 1 is a block diagram of a communication network for responding to an emergency situation according to various embodiments of the disclosure.

FIG. 2 is a diagram illustrating the collection of patient data at an emergency scene using the communication network of FIG. 1 according to various embodiments of the disclosure.

FIGS. 3A, 3B, and 3C are diagrams illustrating the use of a predictive model to optimize emergency vehicle to healthcare facility transport and communications using the patient data collected in FIG. 2 according to various embodiments of the disclosure.

FIG. 4 is a diagram illustrating a method of transmitting medical data associated with the patient through the communication network of FIG. 1 according to various embodiments of the disclosure.

FIG. 5 is a flowchart of a first method of optimizing emergency vehicle to healthcare facility transport and communications in the communication network of FIG. 1 according to various embodiments of the disclosure.

FIG. 6 is a flowchart of a second method of optimizing emergency vehicle to healthcare facility transport and communications in the communication network of FIG. 1 according to various embodiments of the disclosure.

FIGS. 7A and 7B are block diagrams illustrating a communication system similar to the communication system of FIG. 1 according to an embodiment of the disclosure.

FIG. 8 is a block diagram of a computer system implemented within the communication system of FIG. 1 according to an embodiment of the disclosure.

### DETAILED DESCRIPTION

It should be understood at the outset that although illustrative implementations of one or more embodiments are illustrated below, the disclosed systems and methods may be implemented using any number of techniques, whether currently known or not yet in existence. The disclosure should in no way be limited to the illustrative implementations, drawings, and techniques illustrated below, but may be modified within the scope of the appended claims along with their full scope of equivalents.

As discussed above, an emergency vehicle with a few medical personnel may arrive at an emergency scene, and evaluate the individuals that are present at the scene and in need of medical attention. The medical personnel may determine that one or more of these individuals (hereinafter referred to as “patients”) are to be stabilized and transported to a proper healthcare facility for further treatment, surgery, and/or advanced medical care. The healthcare facility may be, for example, a hospital emergency department, trauma center, cardiac center, stroke center, maternity hospital, psychiatric hospital, rehabilitation center, specialty hospital, urgent care center, long-term care facility, etc. The healthcare facility may be owned and/or operated by the same entity as the emergency vehicle, or the emergency vehicle may be owned and/or operated by an entity independent of a local healthcare facility.

Once the patient is stabilized, the patient may be moved into and secured on a platform or seat in the emergency vehicle. The medical personnel or driver of the vehicle may then quickly identify a healthcare facility to send the patient. For example, the driver of the emergency vehicle may simply route to the hospital from the emergency scene. In another example, the driver of the emergency vehicle may route to the closest healthcare facility that is owned and operated by the same entity that owns the emergency vehicle. As another example, the medical personnel may review a map displayed on a display of the emergency vehicle (either in a built-in device or a portable user device) indicating nearby healthcare facilities of different types. The medical personnel may manually select a healthcare facility to transport the patient to based on a location of the healthcare facility and a specialty of the healthcare facility. For example, the manually selected healthcare facility may be the closest cardiac care facility to the emergency scene if the patient is in need of cardiac care. The emergency vehicle may then begin transit to the selected healthcare facility.

During transit, the medical personnel may receive identification data of the patient (e.g., receive/find a government issued identification document of the patient) and input the identification data of the patient into a device at the emergency vehicle. The device may be a computer system with an input device and a display positioned within the emergency vehicle or a mobile device (e.g., cell phone, tablet). For example, the medical personnel may manually input, via the device, data associated with the identification of the patient, stabilization of the patient (e.g., procedures performed to stabilize the patient), and/or treatments performed on the patient prior to the patient being moved into the emergency vehicle and during transit to a healthcare facility. The device may transmit this data back to a healthcare facility system associated with the identified healthcare facility. Medical data collected from various medical devices in the emergency vehicle may also be transmitted to the healthcare facility system.

However, there are numerous technical problems that may occur during the aforementioned process of admitting a patient into an emergency vehicle and transmitting the patient to a healthcare facility (e.g., technical problems that may occur during the process of receiving and processing patient data, identifying an appropriate healthcare facility to route the patient, identifying tests and procedures to perform on the patient during transit, etc.). For example, the medical personnel routes the patient to the nearest healthcare facility without any knowledge regarding the resource capacity at the healthcare facility (physician, staff, and equipment), traffic or congestion on the route to the nearest healthcare facility, the expertise available at the healthcare facility, etc. Instead, the patient may be routed to the nearest healthcare facility, which is often the nearest hospital with an emergency department. In addition, the medical personnel may not have access to any medical history information or current physician information of the patient during transit to the healthcare facility. Instead, the medical personnel merely attempt to keep the patient stabilized during transit.

Lastly, the data from the different medical devices in the emergency vehicle may be sent to the healthcare facility system of the identified healthcare facility in a non-prioritized manner. For example, the data is transmitted over the network to the healthcare facility in the same manner as data from other non-emergency user devices, such that the data may be subject to standard network problems (e.g., congestion, latency, etc.). Each of the technical problems mentioned above may result in system inefficiencies (e.g., delays in the transmission of data related to a patient), reduced reliability in the transmission of the medical data from the medical devices, security risks involved in the transmission of all patient data and medical data, and increased networking and processing resource usage.

The present disclosure addresses the foregoing technical problems by providing a technical solution in the technical field of data management and transport, specifically in the healthcare industry. The embodiments disclosed herein are directed to use of a more comprehensive data set to identify an optimal healthcare facility to route the patient to in the emergency vehicle (in some cases, using an artificial intelligence (AI) model). For example, the comprehensive data set may include identification data of the patient, a current condition of the patient, historical medical data of the patient, healthcare facility expertise data, and/or healthcare facility resource capacity data. That is, instead of always selecting the healthcare facility that is closest to the emergency scene, the systems disclosed herein intelligently select a healthcare facility based on a variety of different of factors indicated in the aforementioned comprehensive data set, particularly when the emergency scene includes many patients, each needing different types of medical attention at different priorities. For example, the systems disclosed herein enable patients that require more immediate medical attention to be routed to the closest hospital, while other patients that are not in need of immediate treatment may be routed to farther hospitals to ensure the closer hospitals have more capacity. In addition, data collected at the emergency vehicle may be transported back to a healthcare facility system associated with the selected optimal healthcare facility in a triaged manner, using network profiles and optimal network paths within network slices, as further described herein.

In some embodiments, the emergency vehicle may include an emergency vehicle system, which may be a computer system communicatively coupled to mobile terminals that the emergency personnel may operate when outside of the emergency vehicle. For example, the emergency personnel may operate tablet devices that may be connected to the emergency vehicle system, and thus may transmit data collected at the emergency scene back to the emergency vehicle system within the emergency vehicle. The emergency vehicle system may include one or more medical devices that may be used to monitor the patient and/or the scene within the emergency vehicle. The medical devices may include, for example, a camera, a sensor, a cardiac monitor, a defibrillator, an oxygen delivery system, computed tomography scanner, a suction unit, airway management equipment, a splinting and immobilization device, first aid supplies, intravenous supplies, diagnostic equipment and/or various types of equipment. The emergency vehicle system may also include a data store that stores medical device data generated/output by each of the medical devices. The emergency vehicle system may also include a vehicle application, that may collect the patient data, retrieve data from the other mobile terminals, display data on a display in the emergency vehicle, transmit data to the healthcare facility system, etc. Each healthcare facility may be associated with a healthcare facility system (e.g., computer system) that may store data relevant to the expertise and resource capacity at each healthcare facility (e.g., physicians, nurses, staff, medical equipment, hospital beds, ventilators, operating rooms, etc.).

After the medical personnel has transported the stabilized patient into the emergency vehicle, secured the patient to the patient platform within the emergency vehicle, and attached monitoring medical devices to the patient, the medical personnel may obtain identification data of the patient. The identification data may include, for example, a full name, date of birth, address, social security number, address, contact information, etc. The medical personnel may ask the patient to provide this information or may find the patient's government issued identification card. The medical personnel may then input the identification data into the emergency vehicle system (e.g., via a user interface). The vehicle application may then obtain (e.g., receive) patient data associated with the patient based on the identification data. For example, the patient data may be stored at a data store, accessible by the vehicle application, such that the vehicle application may request the patient data for a patient using the identification data of the patient. The patient data may include historical patient data describing, for example, at least one of a diagnosis history of the patient, a treatment history of the patient, physician data indicating contact information of one or more current physicians of the patient, or allergy data identifying one or more allergies of the patient.

The emergency vehicle system may receive medical data from the medical personnel and the medical devices in the system. The medical data may include medical device data received from the different medical devices in the vehicle and current patient condition data manually input into the system by the emergency personnel. For example, the medical data may include vital signs (e.g., heart rate, blood pressure, respiratory rate, temperature) (e.g., collected by medical devices), data describing a current state/condition of the patient (e.g., currently experienced symptoms, nature and severity of pain/discomfort, etc.), data based on a brief neurological assessment evaluating the patient's motor function, sensory perception, and cognitive status, etc. The vehicle application may obtain the medical data and input the medical data into a predictive model (e.g., AI model, machine learning model, neural network model, etc.) to make various determinations, including identifying an optimal healthcare facility to transmit the patient.

The predictive model may be trained based on known data and outcomes to make various types of predictions on behalf of the medical personnel in the emergency vehicle, as further described herein. The predictive model may be continuously updated with the expertise and resource capacity data from the different healthcare facility systems (e.g., associated with the different healthcare facilities within a particular region accessible by the emergency vehicle). For example, the predictive model may maintain location data for each healthcare facility, and may also maintain up-to-date data regarding resources available at the healthcare facility, available physicians/nurses/staff of various specialties, operating room schedules, etc. The predictive model may also be continuously updated with route traffic data indicating real-time road conditions, traffic congestions, and/or potential obstacles that may be encountered along a route to each of the healthcare facilities.

When the vehicle application inputs the medical data into the predictive model, the vehicle application may determine an optimal healthcare facility for the patient using the predictive model. The predictive model may run based on the AI and machine learning algorithms programmed at the predictive model to predict an optimal healthcare facility to send the patient based on the inputted medical data, patient data (including historical medical data of the patient), the healthcare facility data, the route traffic data, and/or any other available data. For example, when there are 10 patients at an emergency scene to be routed to a healthcare facility, the predictive model may determine that two patients may need to be routed to the closest hospital, two patients may be routed to another hospital (not necessarily the closest hospital), another patient may be routed to a hospital that is frequency visited by the patient, etc. As another example, the predictive model may determine that, when there is traffic along the route to the closest hospital, that the two high-urgency patients may be routed to the second closest hospital, the route to which is uncongested and clear.

After the vehicle application identifies the optimal hospital, the route to the optimal hospital may be displayed to the driver of the emergency vehicle (e.g., at the navigation/information console of the vehicle, at a display in the vehicle, etc.). The driver of the emergency vehicle may begin transit to the identified hospital. During transit, the medical personnel may continue to monitor the patient, identify the patient, and treat symptoms of the patient, and begin treatment of the patient when appropriate. The vehicle application may provide suggestions for additional test and potential treatment plans (e.g., medicines, oxygen administration, addition scans, etc.) for the patient using the predictive model based on the inputted medical data and the patient data (including historical medical data of the patient). For example, the vehicle application may recommend, using the predicted model, that the patient has a cardiac care history, and that the medical personnel monitor the cardiac vitals of the patients using cardiac equipment (e.g., EKGs).

During transit, the vehicle application may also identify, using the predictive model, physicians that have been in the process of treating/working with the patient for ongoing medical care. For example, the patient may be undergoing cardiac treatment with a cardiologist after a recent cardiac incident, and the vehicle application may obtain details of the cardiac incident/treatment and the contact information of the cardiologist of the patient, and then present this information to the medical personnel on a display. The medical personnel may then review the information and contact the cardiologist (e.g., via videoconference) to receive more detailed instructions for the patient care.

The vehicle application may also request reservation of relevant facility resources at the healthcare facility using the predictive model during transit. The predictive model may run based on the AI and machine learning algorithms programmed at the predictive model to predict resources (e.g., ventilator, surgical operating room, etc.) that may be used on the patient once the patient arrives at the healthcare facility. For example, the identified healthcare facility may already be known to have the capacity for the patient, but this step may allow the medical personnel to reserve an operating room if the determination is made that the patient is to have an emergency surgery.

In an embodiment, the medical data, including the medical device data, may be transmitted in a secure manner to the healthcare facility system associated with the identified healthcare facility in a prioritized and triaged manner. This may be performed using network slices and associated network profiles. A network slice is a virtualized, isolated portion of a network infrastructure, providing a specific set of resources and services tailored to meet requirements of particular user groups, applications, or services. Each network slice may be associated with a network profile. A network profile indicates the requirements, capabilities, and attributes of each associated network slice (e.g., quality of service, service level agreements, resource allocation, security policies, traffic management, service dependencies, etc.).

In an embodiment, each medical device may be associated with a different network profile (and thus a different network slice), different types of medical data may be associated with different network profiles, different attributes related to the data being transmitted (e.g., protocols, ports, etc.) may be associated with different network profiles, etc. The system may maintain different policies indicating the network for different types of data and different types of medical devices. Each network profile may be associated with a different set of resources that may align with a different set of network attribute requirements. For example, videoconferencing data may be associated with a network profile specifying a network attribute requirement for a high-resolution bandwidth, scanned images may be associated with a network profile specifying a network attribute requirement for a high bandwidth and low latency, temperature data may be associated with a network profile with lower baseline network attribute requirements, etc. As another example, diagnostic test results may be associated with a network slice having a low latency requirement, vital signs may be associated with a network slice having a high throughput, patient identification data may be associated with a lower priority network slice, and/or emergency situation data describing a criticality of the patient may be associated with a highest priority/highest network requirement network slice. Each type of data (which may be identified in various different manners) may be associated with different network attributes, and thus a different network profile (and corresponding network slice) that meets the network attributes.

The vehicle application may use the policies to determine the appropriate network profile (and corresponding network slice) for medical data that is to be transmitted to the healthcare facility system. The vehicle application may also determine a network path of elements along the corresponding network slice by which to transmit the medical data. The vehicle application may instruct a network element (e.g., radio transceiver, virtual private network (VPN), virtual network function (VNF)) in the emergency vehicle system to route the medical data along the determined network path in the network slice.

In this way, the embodiments disclosed herein serve to address the technical problems mentioned above, by increasing the efficiency of the system (reducing data transmission delays, prioritizing transmission of high priority data, etc.), increasing the reliability of data transmission in the network slices, increasing the security of data transmission in network slices, and generally decreasing the network and processing usage at the system. Therefore, in general, the embodiments disclosed herein also serve to increase the capacity of the system for medical data transmission using predictive models.

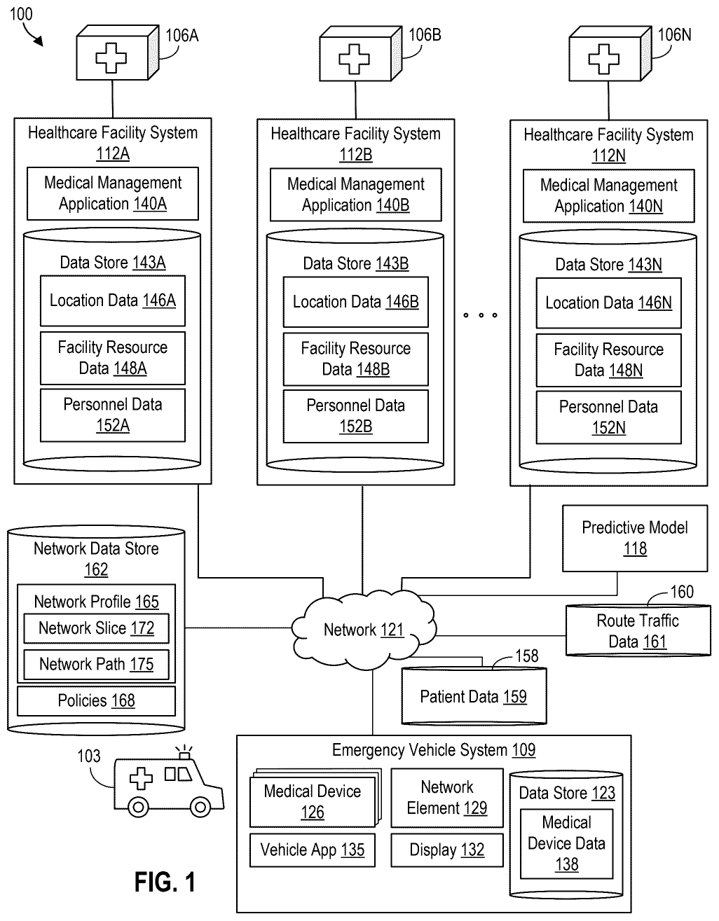

Turning now to FIG. 1, a communication network 100 is described. FIG. 1 illustrates an emergency vehicle 103 and one or more healthcare facilities 106A-N. Each healthcare facility 106A-N may be, for example, a hospital emergency department, trauma center, cardiac center, stroke center, maternity hospital, psychiatric hospital, rehabilitation center, specialty hospital, urgent care center, long-term care facility, etc. Each healthcare facility 106A-N may be owned and/or operated by the same entity as the emergency vehicle 103, or may be owned and/or operated by an entity independent of an emergency vehicle 103.

The emergency vehicle 103 may include an emergency vehicle system 109, and each healthcare facility 106A-N may be associated with a corresponding healthcare facility system 112A-N. The communication network 100 of FIG. 1 includes the emergency vehicle system 109, the healthcare facility systems 112A-N, a predictive model 118, a patient data store 158, a route traffic data store 160, a network data store 162, and a network 121. The network 121 may be one or more private networks, one or more public networks, or a combination thereof. While FIG. 1 shows the emergency vehicle system 109, healthcare facility systems 112A-C, predictive model 118, patient data store 158, route traffic data store 160, and network data store 162 as being separate from the network 121, it should be appreciated that one or more of the emergency vehicle system 109, healthcare facility systems 112A-C, predictive model 118, patient data store 158, route traffic data store 160, and network data store 162 may be part of the network 121.

The emergency vehicle system 109 and the healthcare facility systems 112A-N may each be connected to the network 121 using a wired or wireless communication link (e.g., using a local area network or a base station, and communicating to the network 121 via a cellular or WiFi connection). For example, the emergency vehicle system 109 and the healthcare facility systems 112A-N may communicate with the network 121 according to a 5G, a long term evolution (LTE), a code division multiple access (CDMA), or a global system for mobile communications (GSM) wireless telecommunication protocol. The network 121 may include a telecommunications access network operated by a telecommunications service provider, a radio access network (RAN), a core network, and/or other network elements.

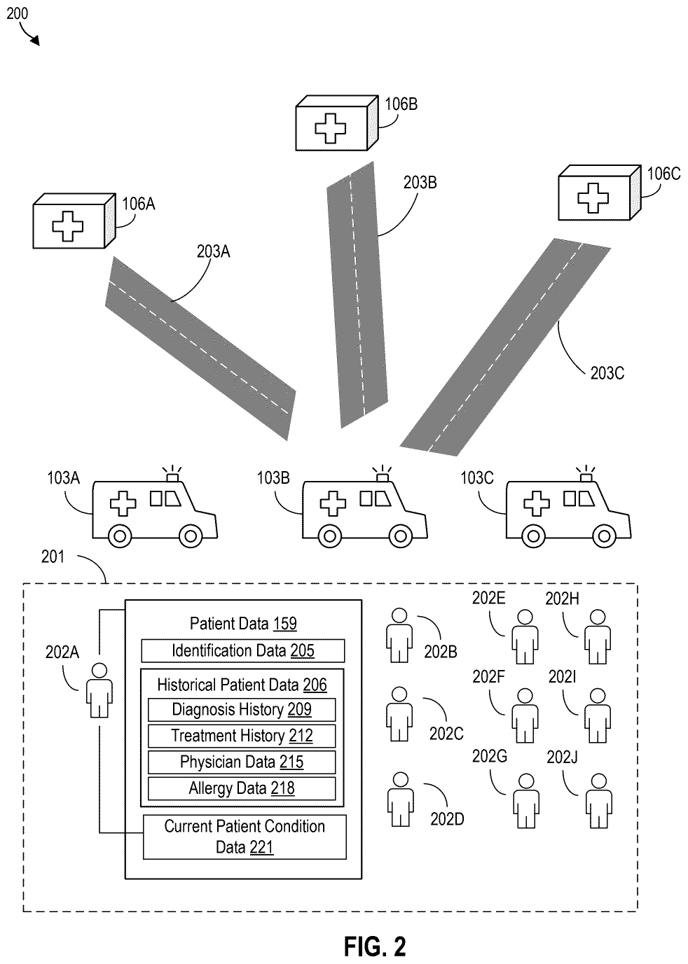

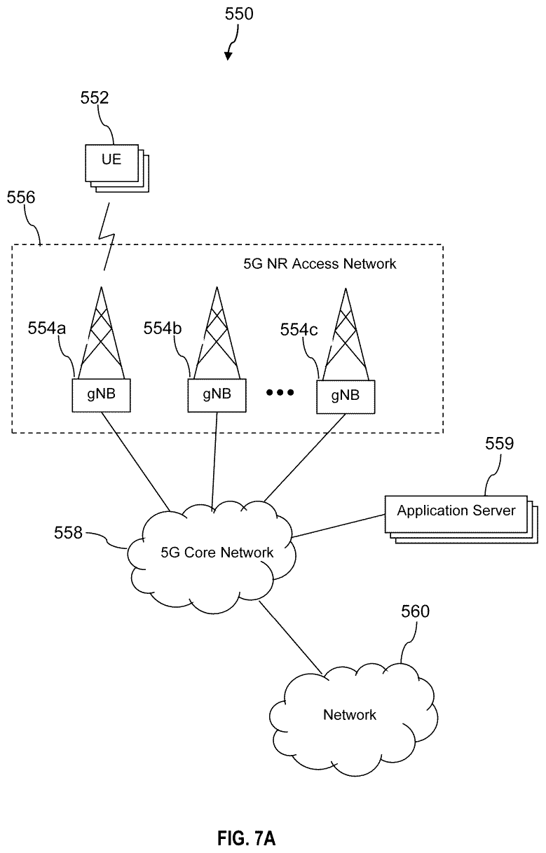

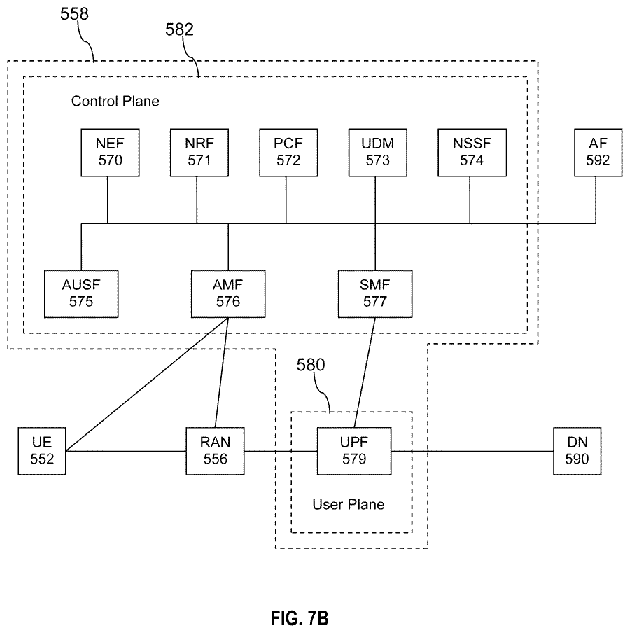

The emergency vehicle system 109 may be a computer system (as further described herein with reference to FIG. 7), with multiple interrelated components, each communicatively coupled to or located in the emergency vehicle 103. The emergency vehicle system 109 may include the medical devices 126, a network element 129, a display 132, and a data store 123. The medical devices 126 may include, for example, a camera, a sensor, a cardiac monitor, a defibrillator, an oxygen delivery system, computed tomography scanner, a suction unit, airway management equipment, a splinting and immobilization device, first aid supplies, intravenous supplies, diagnostic equipment, and/or any other type of medical equipment. The network element 129 may be, for example, a router, switch, VPN, VNF, etc., configured to instruct the routing of medical data according to network profiles, as further described herein. The display 132 may be configured to display different types of information to the medical personnel, and/or may be configured to display a route to a healthcare facility 106A-N (e.g., in a navigation console). The data store 123 may include one or more memories for storing medical device data 138, which may be collected by all the different medical devices 126 in the emergency vehicle system 109. For example, the medical device data 138 may include videoconferencing data associated with videoconferences occurring in the emergency vehicle 103, real-time vital sign monitoring data, ongoing assessment findings, medical procedure data, patient condition data, image data associated with images received from cameras deployed in the emergency vehicle 103, etc. The emergency vehicle system 109 may also include a vehicle application 135, which may be instructions stored on a memory of the emergency vehicle system 109. When the vehicle application 135 is executed by a processor at the emergency vehicle system 109, the vehicle application 135 may perform the steps and operations as further described with reference to FIGS. 2-6.

Each healthcare facility system 112A-N may be a computer system, server software/hardware, or a collection of processors, memories, and/or networking resources, used to manage, receive, and transmit different types of data as described herein. For example, each healthcare facility system 112A-N may be embodied as a cloud-based system, which may include one or more data stores and memories located together or separately across geographically disparate locations, separate from the respective healthcare facility 106A-N. Each healthcare facility system 112A-N may also be embodied as a local set of data stores and memories positioned within or proximate to the respective healthcare facility 106A-N.

Each healthcare facility system 112A-N may include a medical management application 140A-N, respectively. The medical management application 140A-N may be instructions stored on a memory of the healthcare facility system 112A-N. When the medical management application 140A-N is executed by a processor at the healthcare facility system 112A-N, the medical management application 140A-N may transmit and receive communications from the vehicle application 135, update the data that may be used to train the predictive model 118 (as further described herein), update the data used by the predictive model 118 to generate predictions/suggestions (as further described herein), etc.

Each healthcare facility system 112A-N may also include a data store 143A-N, respectively. The data stores 143A-N may maintain data describing attributes associated with the respective healthcare facility system 112A-N. As shown in FIG. 1, the data store 143A-N stores location data 146A-N, facility resource data 148A-N, personnel data 152A-N, and other data not otherwise shown in FIG. 1 or described herein. The location data 146A-N may identify a location of the respective healthcare facility 106A-N. The facility resource data 148A-N may identify the resource capacity at the respective healthcare facility 106A-N (e.g., open hospital beds/rooms, emergency department beds, ventilators, infusion pumps, pharmaceuticals, etc.). The personnel data 152A-N may describe the availability and expertise of the individuals employed at the respective healthcare facility 106 (e.g., number of available cardiothoracic surgeons, number of available hospitalists, number of available emergency medical physicians, number of available senior nurses, number of available administrative personnel, experience/education level of the physicians, etc.).

The communication network 100 may include data stores 158, 160, and 162 storing different types of data not only used to perform the methods disclosed herein, but also to train the predictive model 118. The patient data store 158 may include patient data 159, including currently collected patient data and historical patient data. The currently collected patient data may be data documented by the medical personnel in the emergency vehicle during the process of stabilizing the patient and during transit, such as, for example, current symptoms experienced by the patient, nature and severity of patient pain/discomfort, etc. (also referred to herein as “current patient condition data”). The historical patient data may include, for example, a diagnosis history of the patient, a treatment history of the patient, physician data indicating information of one or more current physicians of the patient, allergy data identifying one or more allergies of the patient, etc.

The route traffic data store 160 includes route traffic data 161. The route traffic data 161 may be a live, continuously updated data feed of real-time road conditions anywhere within the vicinity, road, highway, or airway path to the healthcare facilities 106A-N. The route traffic data 161 may indicate traffic congestions or potential obstacles that may be encountered along a route to the healthcare facilities 106A-N.

The network data store 162 may store data regarding the network profiles 165 assigned to different types of medical data (e.g., medical device data 138 and/or patient data 159 collected at the emergency vehicle 109). The network profiles 165 may each include an identification of an associated network slice 172 and the tailored resources/services provided by the associated network slice 172. The network profiles 165 may also include one or more optimal paths to different destination healthcare facility systems 112A-N using the resources within the associated network slice 172. The network data store 162 may also include the policies 168, which may indicate the associations between the different types of medical devices 126, medical device data 138, and patient data 159 and the associated network profile 165 (e.g., a policy 168 may indicate that medical device data 138 from a particular medical device 126 is assigned a particular network profile 165).

The predictive model 118 may be a computer system (e.g., including both software and hardware components) designed to make predictions or forecasts (e.g., the optimal healthcare facility 112A-N for a patient, suggested treatments, recommended resource reservations at a healthcare facility 112A-N, current physician contact information, etc.) based on patterns or trends learned from historical data (e.g., patient data 159, medical device data 138, route traffic data 161, etc.). The predictive model 118 may be implemented using software (e.g., algorithms, logic, and code) stored across memories. The predictive model 118 may be hosted on a standalone server (separate from the healthcare facility systems 112A-N and the emergency vehicle system 109), or the predictive model 118 may be hosted within the healthcare facility systems 112A-N and/or the emergency vehicle system 109. The host of the predictive model 118 may provide the computational resources for execution of the predictive model 118.

The predictive model 118 may be implemented as one or more different types of models using, for example, linear regression, decision trees, support vector machines, neural networks, or ensemble methods. The predictive model 118 may be a machine learning model, deep learning model, neural networking model, natural language processing (NLP) model, or any other type of AI model. It should be appreciated that any type of AI/predictive model may be used, and the underlying algorithms, computations, and machine learning libraries used by the predictive model 118 should not be limited herein.

The predictive model 118 may be trained using various types of known data and outcomes. For example, the predictive model 118 may be trained using the known data from the data stores 143A-N, data store 123, patient data store 158, and/or route traffic data store 160 such that the data points and algorithms in the predictive model 118 may be used to identify patterns/trends to predict optimal healthcare facility systems 112A-N for patients, recommend resource reservations at the healthcare facilities 106, identify potential treatment plans and/or additional tests to be performed on the patient while in transit, etc.

Referring now to FIG. 2, shown is a diagram 200 illustrating an emergency scene 201 (e.g., location of a natural/man-made disaster or accident), at which multiple patients 202A-J may be in need of medical attention. In the example shown in FIG. 2, three emergency vehicles 103A-C have arrived at the emergency scene 201, and there are three healthcare facilities 106A-C within a predefined distance from the emergency scene 201. FIG. 2 also illustrates routes 203A-C routing from the emergency scene 201 to the healthcare facilities 106A-C. The routes 203A-C are each shown as single, two-lane roads for illustrative purposes only, and it should be appreciated that the routes between emergency scenes 201 and healthcare facilities 106A-C may involve different directions, turns, and multiple roads. It should be appreciated that the emergency scene 201 may include any number of patients 202A-J, any number of emergency vehicles 103A-C may arrive at an emergency scene 201, and there may be any number of healthcare facilities 106A-C within the predefined distance range from the emergency scene 201.

The medical personnel from the emergency vehicles 103A-C may arrive at the emergency scene 201 and approach the patients 202A-J individually (based on need). The medical personnel may be carrying a mobile device that automatically pushes all data input at the mobile device back to the emergency vehicle system 109 of the associated emergency vehicles 103A-C. In some cases, mobile devices from the medical personnel associated with each of the three different emergency vehicles 103A-C may push data to either the associated emergency vehicle 103A-C or the emergency vehicle system 109. In this way, the mobile devices of the medical personnel may share data across the emergency vehicles 103A-C that approached the emergency scene 201.

The medical personnel may input patient data 159 into the mobile device at the time of interacting with each patient 202A-J. The patient data 159 may include the identification data 205 identifying the patient 202A-J. The patient data 159 may also include current patient condition data 221 (e.g., current symptoms, pain level, blood pressure, temperature, oxygen level, etc.) indicative of a current state/condition of the patient 202A-J.

The patient data 159 may be sent to one or more of the emergency vehicles 103A-C and/or directly to the emergency vehicle system 109. The mobile device (and/or the emergency vehicle system 109) may in some cases use the identification data 205 of the patient to obtain historical patient data 206 associated with the patient 202A-J (e.g., from the patient data store 158). The historical patient data 206 may include, for example, a diagnosis history 209 (e.g., previously diagnosed conditions of the patient 202A-J), a treatment history 212 (e.g., previous treatments, procedures, surgeries, etc. undergone by the patient 202A-J), physician data 215 (e.g., prior and current physicians of the patient 202A-J, contact information for the physicians), allergy data 218 (e.g., identifying one or more allergies of the patient 202A-J), and/or any other historical data applicable to the health of the patient 202A-J.

The medical personnel may obtain the patient data 159 for each patient 202A-J at the emergency scene 201 or once the patient 202A-J has been moved into an emergency vehicle 103A-C. The medical personnel may repeat the process of collecting patient data 159 for all of the different patients 202A-J at the emergency scene 201. In an embodiment, patients 202A-J may be routed to different healthcare facilities 106A-C based on various factors, such as, for example, the severity of the patient 202A-J (e.g., determined based on patient data 159), the distance between the emergency scene 201 and the healthcare facilities 106A-C (e.g., determined based on location data 146A-N at the healthcare facility systems 112A-N), and resources available at the healthcare facilities 106A-C (e.g., determined based on the facility resource data 148A-N and/or personnel data 152A-N).

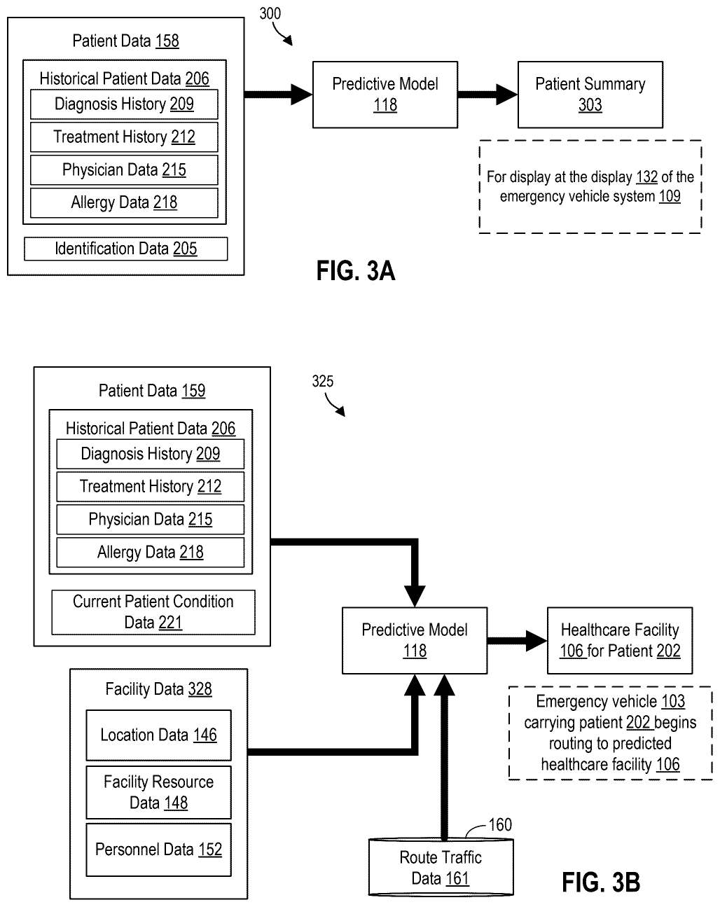

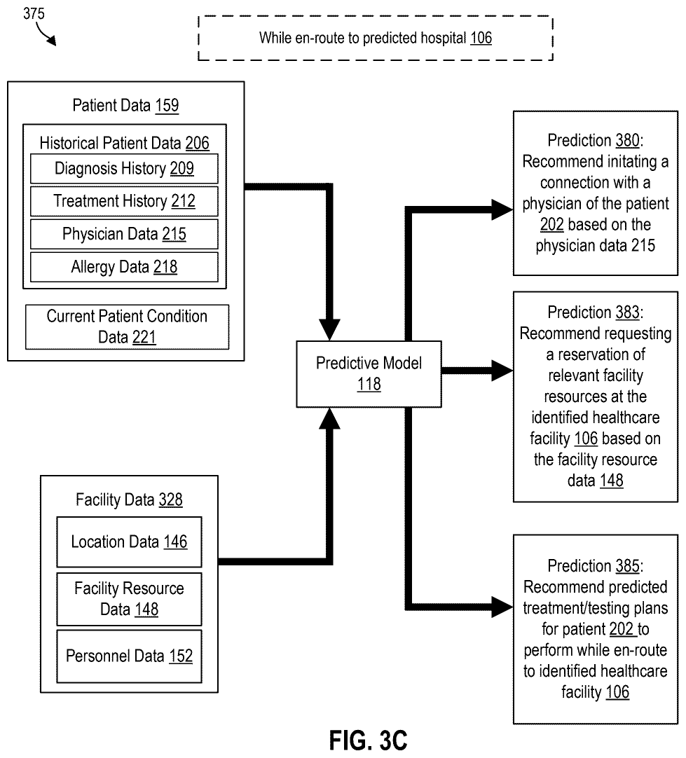

Referring now to FIGS. 3A, 3B, and 3C, shown are diagrams illustrating the use of the predictive model 118 in optimizing emergency vehicle 103A-C (hereinafter referred to as “emergency vehicle 103”) to healthcare facility 106A-N (hereinafter referred to as “healthcare facility 106”) transport and communications. Specifically, FIG. 3A illustrates the use of the predictive model 118 to generate a patient summary, FIG. 3B illustrates the use of the predictive model 118 to identify an optimized healthcare facility 106 for a patient, and FIG. 3C illustrates the use of the predictive model 118 to determine additional predictions/recommendations on behalf of the medical personnel in the emergency vehicle 103.

Turning now to FIG. 3A, shown is the use of the predictive model 118 to generate a patient summary 303. The patent summary 303 may be a concise overview of a medical history of the patient and relevant health information of the patient, and the patient summary 303 may be primarily based on the historical patient data 206.

The medical personnel may first obtain (e.g., receive) the identification data 205 of the patient and input the identification data 205 into the emergency vehicle system 109 (or a mobile device connected to the emergency vehicle system 109). The vehicle application 135 at the emergency vehicle system 109 may use the identification data 205 to obtain (e.g., retrieve) the historical patient data 206 associated with the patient from the patient data store 158. For example, the vehicle application 135 may transmit a request to the patient data store 158 for the patient data 159 or specifically the historical patient data 206 associated with the identification data 205 of the patient, and the patient data store 158 may transmit the patient data 159 or the historical patient data 206 of the patient back to the vehicle application 135. The vehicle application 135 may input the patient data 159, including the identification data 205 of the patient, the retrieved historical patient data 206, and current patient condition data 221 (manually collected by the medical personnel), into the predictive model 118. In this process, the medical personnel may not have to review the historical patient data 206, since this data 206 may be extensive and include complex information/images, some of which may not be relevant in the current context. The predictive model 118 may be trained to identify the relevant and significant patterns of data from the historical patient data 206 (e.g., the diagnosis history 209, treatment history 212, physician data 215, allergy data 218, etc.) based on the current patient condition data 221. For example, if the current injury to the patient is at the knee of the patient, the predictive model 118 may intelligently obtain (e.g., extract) all historical patient data 206 applicable to the knees of the patient, all knee/orthopedic physicians of the patient, allergy data 218 related to medicines used to treat knee injuries, etc.

The predictive model 118 may output a patient summary 303 indicating the obtained relevant and significant items of data from the historical patient data 206 based on the current patient condition data 221. The predictive model 118 may output the patient summary 303 using a form of generative AI, such that the patient summary 303 is a concise, easy to read paragraph or bullet point summary of the relevant and significant items of data obtained based on an analysis of the historical patient data 206 using the current patient condition data 221. The patient summary 303 may be displayed at the display 132 of the emergency vehicle system 109 prior to administering any medicines, performing additional tests, or providing treatment to the patient.

Turning now to FIG. 3B, shown is the use of the predictive model 118 to predict the optimal healthcare facility 106 for the patient. For example, after the medical personnel has reviewed the patient summary 303, obtained additional information from the patient (if the patient is conscience), and/or requested a recommended optimized healthcare facility 106 for the patient (e.g., by selecting an icon on a user interface displayed on display 132 at the emergency vehicle system 109), the vehicle application 135 may access the predictive model 118 to predict the optimal healthcare facility 106 for the patient. The vehicle application 135 may provide the patient data 159 as input into the predictive model 118. The patient data 159 may include the historical patient data 206 (e.g., diagnosis history 209, treatment history 212, physician data 215, allergy data 218, etc.) and current patient condition data 221.

As mentioned above, the predictive model 118 may have access to and/or maintain updated data from all of the different healthcare facilities 106 available within a predefined distance from the location of the emergency vehicle 103 (e.g., emergency scene 201). The predictive model 118 may also have access to and/or maintain up-to-date route traffic data 161 from the route traffic data store 160. When the predictive model 118 receives the patient data 159 as input from the vehicle application 135, the predictive model 118 may obtain (e.g., access, retrieve, receive, search) the facility data 328 and the route traffic data 161 to the healthcare facilities 106 within a predefined distance from the emergency vehicle 103. The facility data 328 may refer to the data stored at the data stores 143A-N (hereinafter referred to as “data stores 143”) in each of the healthcare facility systems 112A-N, including the location data 146A-N (hereinafter referred to as “location data 146”), facility resource data 148A-N (hereinafter referred to as “facility resource data 148”), and personnel data 152A-N (hereinafter referred to as “personnel data 152”). In this way, the facility data 328 and the route traffic data 161 may be considered input into the predictive model 118 (even though the vehicle application 135 may not actively send the facility data 328 and route traffic data 161 to the predictive model 118).

In another embodiment, the vehicle application 135 may obtain (e.g., receive, retrieve, etc.) the facility data 328 from the different healthcare facility systems 112A-N within the predefined distance of the current location of the emergency vehicle 109 (e.g., the emergency scene 201), and may obtain the route traffic data 161 from the route traffic data store 160. The vehicle application 135 may then send this data to the predictive model 118 with the patient data 159 as input.

The predictive model 118 may be programmed with one or more different types of machine algorithms, which have been adequately trained as described above, to select an optimal healthcare facility 106 for the patient from all of the healthcare facilities 106 within the predefined distance of the current location of the emergency vehicle 103. The prediction model 118 may select the healthcare facility 106 for the patient based on the patient data 159, the facility data 328, and the route traffic data 161. For example, the prediction model 118 may select the healthcare facility 106 for the patient based primarily on an evaluation of the current patient condition data 221 (e.g., indicating a severity of the condition of the patient, symptoms experienced by the patient, whether surgery may be needed for the patient, etc.) relative to the facility data 328 (e.g., the physicians/resources available at the healthcare facility 106, the location of the healthcare facility 106), and based on the route traffic data 161 (e.g., whether the route from the current location to the healthcare facility 106 is not congested and free of traffic and obstacles). For example, the algorithms in the predictive model 118 may assign higher weights in the algorithm to the current patient condition data 221 and facility data 328, to indicate the importance of these two factors in determining the healthcare facility 106 for the patient.

In this way, the predictive model 118 may use a robust, comprehensive set of data to predict an optimal healthcare facility 106 to route the patient to, without the medical personnel or the driver of the emergency vehicle 109 taking the time or energy to make this determination. The determined optimal healthcare facility 106 may be able to provide better treatment for the patient, given the optimized resources at the healthcare facility 106 that are determined by the prediction model 118 to meet the needs of the patient. Therefore, the embodiments directed to predicting the optimal healthcare facility 106 may more efficiently and effectively use healthcare resources, directly providing for faster and better quality of medical treatment.

Turning now to FIG. 3C, shown is the use of the predictive model 118 to make various predictions 380, 383, and 385 for the patient. The predictions 380, 383, and 385 may be made after the optimal healthcare facility 106 is determined for the patient, while the patient is in transit to the healthcare facility 106. During this time when the patient is in transit to the healthcare facility 106, the medical personnel may have to continuously monitor the patient (e.g., using the medical devices 126), perform regular assessments and reassessments (e.g., conduct physical examinations, observing patient responsiveness, evaluating changes to the patient condition, etc.), perform interventions and treatments (e.g., administering medications, providing oxygen therapy, controlling bleeding, etc.), communicating with dispatch at the receiving healthcare facility 106, documentation, patient care and comfort, etc. However, the medical personnel in the emergency vehicles 103 are not always trained physicians that have gone through the full medical training to provide optimal treatment and care to patients (though the medical personnel in the emergency vehicles 103 have met the training and certification requirements). Therefore, the embodiments disclosed herein may use the predictive model 118 to provide medicine-based, tested medical recommendations to the medical personnel, such that the medical personnel may make more informed decisions while performing the aforementioned tasks and operations on the patient during transit.

As described above in FIG. 3B, the medical personnel may have already input current patient condition data 221 into the emergency vehicle system 109, and the vehicle application 135 may have already provided the patient data 159 (including the historical patient data 206 and the current patient condition data 221) to the predictive model 118. The predictive model 118 may also receive the facility data 328 describing the healthcare facilities 106 as input (either by retrieving the facility data 328 from the different healthcare facility systems 112A-N or from the vehicle application 135 providing the facility data 328 as input into the predictive model 118). Therefore, the relevant inputs may have already been provided to the predictive model 118 at the time of determining the optimal healthcare facility 106 for the patient. The predictive model 118 may then determine the predictions 380, 383, and 385 after or simultaneously with the determining of the optimal healthcare facility 106.

The predictive model 118 may be programmed with one or more different types of machine algorithms, which have been adequately trained as described above, to make various predictions 380, 383, and 385 based on the input data. For example, the predictive model 118 may output the prediction 380 to recommend initiating a connection with a physician of the patient. This prediction 380 may be primarily based on the physician data 215 in the historical patient data 206. In some cases, the physician data 215 may be assigned a higher weight in the algorithms of the predictive model 118. For example, the patient may be undergoing cardiac treatment with a cardiologist after a recent cardiac incident, which may be indicated in the physician data 215. The predictive model 118 may use the current patient condition data 221 to determine whether cardiac care may be relevant to the patient. When the expertise or opinion of the physician (e.g., the cardiac care) is relevant to the patient during the transit to the healthcare facility 106 based on the current condition of the patient, the predictive model 118 may output the prediction 380 to recommend contacting the physician (e.g., the cardiologist) of the patient. The predictive model 118 may output the prediction 380 using a form of generative AI, such that the prediction 380 is a concise, easy to read statement with the relevant physician details, prior care from the physician, and contact information of the physician. The vehicle application 135 may display the generated prediction 380 at the display 132 of the emergency vehicle system 109.

In addition, the predictive model 118 may output the prediction 383 to recommend requesting a reservation of relevant facility resources at the identified optimal healthcare facility 106. This prediction 383 may be primarily based on the facility resource data 148 in the facility data 328 and the current patient condition data 221. The facility resource data 148 in the facility data 328 and the current patient condition data 221 may be assigned higher weights in the algorithm of the predictive model 118. For example, the medical personnel may determine that the patient needs a ventilator upon arriving at the healthcare facility 106, and the predictive model 118 may determine other resources/medical equipment that may also be needed by the patient after arriving at the healthcare facility, based on the current patient condition data 221. The prediction 383 may include a recommendation to reserve the additional determined resources/medical equipment, and even an option to instruct the vehicle application 135 to send a request to the healthcare facility 106 to request reservation of the resources/medical equipment. The predictive model 118 may output the prediction 383 using a form of generative AI, such that the prediction 383 is a concise, easy to read statement describing the additional determined resources/medical equipment to request. The vehicle application 135 may display the generated prediction 383 at the display 132 of the emergency vehicle system 109.

The predictive model 118 may also output the prediction 385 to recommend predicted treatment/testing plans for the patient for the medical personnel to perform while en-route to the healthcare facility 106. This prediction 385 may be relatively equally based on the diagnosis history 209 and/or treatment history 212 in the historical patient data 206 and the current patient condition data 221. For example, predictive model 118 may have been trained using historical data indicative of patients having certain symptoms and conditions, helpful tests that were performed on the patients, and treatments/medicines that were administered to the patients that helped relieve the symptoms and further stabilize the patient until the patient reached the destination healthcare facility 106. The predictive model 118 may thus be trained to use the input data (e.g., patient data 159 including the historical patient data 206 and current patient condition data 221) to predict additional helpful tests to perform on the patients, medicines to be administered to the patients, treatment plans/procedures to perform on the patient. The predictive model 118 may output the prediction 385 using a form of generative AI, such that the prediction 385 is a concise, easy to read statement with the data and instructions regarding the recommended tests to be performed on the patient, recommended medicines to be administered to the patient, and/or recommended treatment plans/procedures to be performed on the patient. The vehicle application 135 may display the generated prediction 385 at the display 132 of the emergency vehicle system 109.

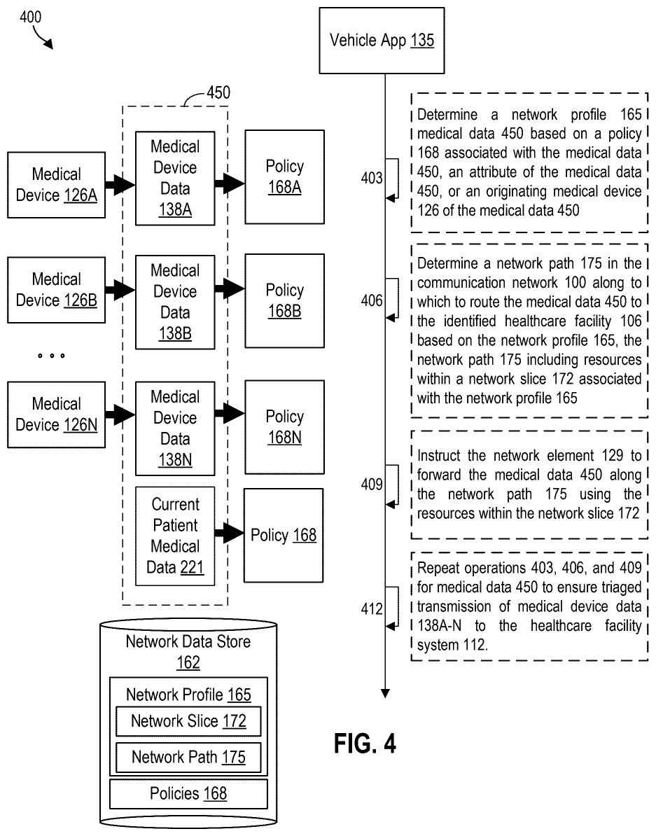

Referring now to FIG. 4, shown is a diagram illustrating a method 400 for transmitting medical data 450 (including patient data 159 and medical device data 138) to the healthcare facility system 112 associated with the identified optimal healthcare facility 106. In an embodiment, the medical data 450 may be transmitted along different network slices 172 based on a network profile 165 assigned to the type of medical data being transmitted. One or more policies 168 may indicate the association between a type of medical data 450 and the corresponding network profile 165. Network slices 172 may be virtualized, isolated portions of a 5G network infrastructure tailored to specific applications, services, or user groups, enabling customized resource allocation and service delivery within a network slice 172. Within 5G core networks, network slices 172 allow for the creation of multiple virtualized network instances, each optimized to meet the diverse requirements of different use cases, providing flexibility, scalability, and efficient management of network resources to provide 5G core network services.

As shown in FIG. 4, each type of medical device 126A-N may generate medical device data 138A-N respectively. The medical device data 138A-N from different types of medical devices 126A-N may each have different attributes associated with the data traffic in the respective medical device data 138A-N (e.g., data type, content, protocols, headers, ports for sending/receiving the data, etc.). Each of the medical devices 126A-N and/or outputted medical device data 138A-N may be associated with a different policy 168A-N. One of policies 168A-N may respectively indicate that medical device data 138A-N is associated with a particular network profile 165 based on at least one of the following: the medical device data 138 originates from a particular type of medical device 126A-N, an attribute of the medical device data 138A-N matches a preset attribute, and/or other factors. For example, a policy 168A-N may indicate that medical device data 138A-N may include video data and may be associated with a network profile 165, which is associated with a particular network slice 172. The network profile 165 may also be used to determine an optimal network path 175 within the network slice 172 to the identified healthcare facility system 112.

Current patient condition data 221, which may be automatically or manually obtained by the medical personnel, and then input into the emergency vehicle system 109, may also be associated with a particular policy 168. The policy 168 may also be based on the type of the current patient condition data 221 (e.g., symptoms, procedures performed, diagnosis, etc.), such that different types of current patient condition data 221 is assigned to different network profiles 165.

The method 400 shown in FIG. 4 may be performed by the vehicle application 135 to identify network profiles 165 for different types of medical data 450 (medical device data 138A-N (hereinafter referred to as “medical device data 138”) and/or current patient condition data 221) based on the policies 168A-N (hereinafter referred to as “policies 168”). At operation 403, the vehicle application 135 may determine a network profile 165 for the medical data 450 based on a policy 168 associated with at least one of the medical data 450, an attribute of the medical data 450, or an originating medical device 126 of the medical data 450. For example, when the policy 168 identifies the type of medical data 450, an attribute of the medical data 450, or an originating medical device 126 of the medical data 450, the policy 168 may be applied to the medical data 140 to determine the network profile 165 assigned to the medical data 140.

At operation 406, the vehicle application 135 may determine a network path 175 in the communication network 100 along which to route the medical data 450 to the healthcare facility system 112 of the identified healthcare facility 106 based on the network profile 165. For example, the network path 175 may include one or more network elements (e.g., routers, switches, VNFS, etc.) in the network slice 172 associated with the network profile 165 (e.g., meeting the network attribute characteristics of the network profile 165). The network path 175 may originate at the location of the emergency vehicle 103 and to route the medical data 450 to the healthcare facility system 112. At operation 409, the vehicle application 135 may instruct the network element 129 to forward the medical data 450 along the network path 175 using the resources within the network slice 172. Operation 412 indicates that operations 403, 406, and 409 may be repeated for the different types of medical data 450 to ensure prioritized and triaged transmission of the different types of medical data 450 to the healthcare facility system 112.

While FIGS. 3A-C discuss the predictive model 118 outputting the patient summary 303, the identification of an optimal healthcare facility 106, and various predictions 380, 383, and 385, it should be appreciated that the vehicle application 135 uses the predictive model 118 to determine the patient summary 303, the identification of an optimal healthcare facility 106, and various predictions 380, 383, and 385 (i.e., the predictive model 118 is the AI model used to make predictions as instructed by the vehicle application 135). In an embodiment, the vehicle application 135 may determine the patient summary 303, the identification of an optimal healthcare facility 106, and various predictions 380, 383, and 385 without the use of the predictive model 118.

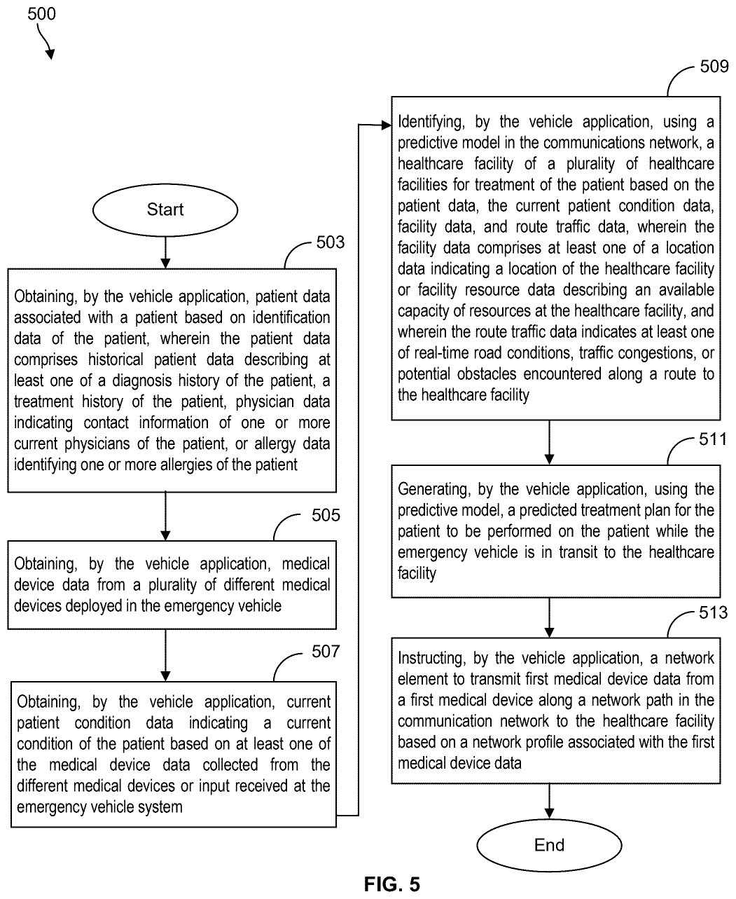

Referring now to FIG. 5, shown is a method 500 for optimizing emergency vehicle 103 to healthcare facility 106 transportation and communications. The method 500 may be implemented in the communication network 100. In embodiments, the method 500 may be implemented using a computer system with components as shown in FIG. 7. As illustrated, method 500 of FIG. 5 includes a number of enumerated operations, but embodiments of the operations in FIG. 5 may include additional operations before, after, and in between the enumerated operations. In some embodiments, one or more of the enumerated operations may be omitted or performed in a different order.

At step 503, method 500 may comprise obtaining, by the vehicle application 135 implemented by the emergency vehicle system 109 in the communication network 100, patient data 159 associated with a patient based on identification data 205 of the patient. The patient data 159 comprises historical patient data 206 describing at least one of a diagnosis history 209 of the patient, a treatment history 212 of the patient, physician data 215 indicating contact information of one or more current physicians of the patient, or allergy data 218 identifying one or more allergies of the patient. At step 505, method 500 may comprise obtaining, by the vehicle application 135, medical device data 138 from a plurality of different medical devices 126 deployed in the emergency vehicle 103. At step 507, method 500 may comprise obtaining, by the vehicle application 135, current patient condition data 221 indicating a current condition of the patient based on at least one of the medical device data 138 collected from the different medical devices 126 or input received at the emergency vehicle system 109.

At step 509, method 500 may comprise identifying, by the vehicle application 135, using a predictive model 118 in the communications network 110, a healthcare facility 106 of the plurality of healthcare facilities 106 for treatment of the patient based on the patient data 159, the current patient condition data 221, facility data 328, and route traffic data 161. The facility data 328 comprises at least one of a location data 146 indicating a location of the healthcare facility or facility resource data 148 describing an available capacity of resources at the healthcare facility 106. The route traffic data 161 indicates at least one of real-time road conditions, traffic congestions, or potential obstacles encountered along a route to the healthcare facility 106.

At step 511, method 500 may comprise generating, by the vehicle application 135, using the predictive model 118, a predicted treatment plan for the patient to be performed on the patient while the emergency vehicle is in transit to the healthcare facility. At step 513, method 500 may comprise instructing, by the vehicle application 135, a network element 129 to transmit first medical device data 138 from a first medical device 126 along a network path 175 in the communication network 100 to the healthcare facility 106 based on a network profile 165 associated with the first medical device data.

Method 500 may include additional steps, operations, and elements not explicitly shown in FIG. 5. In an embodiment, method 500 may further comprise obtaining, by the vehicle application 135, the identification data 205 of the patient to be transported to one of a plurality of healthcare facilities 106 for treatment. For example, the medical personnel may enter, via a user interface of a mobile device connected to emergency vehicle system 109, the identification data 205 of the patient. The mobile device may transmit the identification data 205 back to the emergency vehicle system 109. In an embodiment, method 500 may further comprise obtaining, by the vehicle application 135, using the predictive model 118, a patient summary 303 associated with the patient based on the patient data 159, in which the patient summary 303 is a concise overview of a medical history of the patient and relevant health information of the patient, and displaying, by the vehicle application 135, the patient summary 303 at a display 132 of the emergency vehicle system 109.

In an embodiment, the medical device data 138 comprises at least one of videoconferencing data associated with videoconferences occurring in the emergency vehicle 103, real-time vital sign monitoring data, ongoing assessment findings, medical procedure data, patient condition data, or image data associated with images received from cameras deployed in the emergency vehicle 103. In an embodiment, while the emergency vehicle 103 is in transit to the healthcare facility 106, method 500 may further comprise generating, by the vehicle application, using the predictive model, a recommendation for reserving relevant facility resources at the healthcare facility 106 based on the facility resource data 148. In an embodiment, while the emergency vehicle 103 is in transit to the healthcare facility 106, method 500 may further comprise generating, by the vehicle application, using the predictive model, a recommendation to initiate contact with a physician of the patient based on the physician data 215, the recommendation including contact information for the physician.

In an embodiment, when the current patient condition data 221 indicates that the patient is in a critical condition and in need of immediate medical care, the identifying the healthcare facility 106 for treatment of the patient is further based on a distance between a current location of the patient and the location of the healthcare facility 106. In an embodiment, when the current patient condition data indicates that the patient needs surgery to treat an injury, the identifying the healthcare facility 106 for treatment of the patient is further based on surgeons and surgical resources available at the healthcare facility 106.

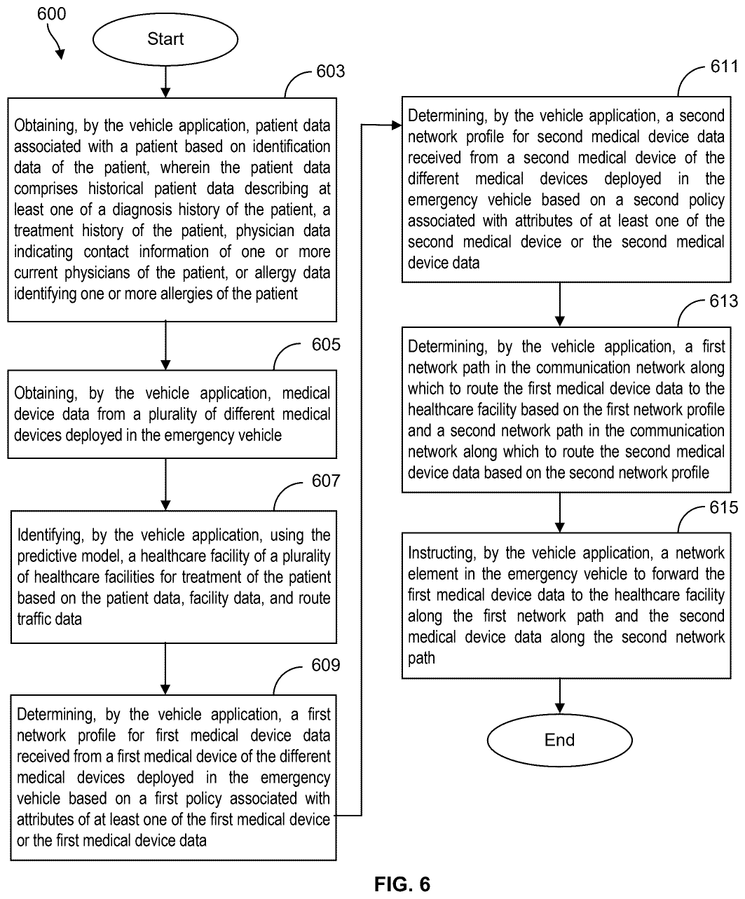

Referring now to FIG. 6, shown is a method 600 for optimizing emergency vehicle 103 to healthcare facility 106 transportation and communications. The method 600 may be implemented in the communication network 100. In embodiments, the method 600 may be implemented using a computer system with components as shown in FIG. 7. As illustrated, method 600 of FIG. 6 includes a number of enumerated operations, but embodiments of the operations in FIG. 6 may include additional operations before, after, and in between the enumerated operations. In some embodiments, one or more of the enumerated operations may be omitted or performed in a different order.

At step 603, method 600 may comprise obtaining, by the vehicle application 135 implemented by the emergency vehicle system 109 in the communication network 100, patient data 159 associated with a patient based on identification data 205 of the patient. The patient data 159 comprises historical patient data 206 describing at least one of a diagnosis history 209 of the patient, a treatment history 212 of the patient, physician data 215 indicating contact information of one or more current physicians of the patient, or allergy data 218 identifying one or more allergies of the patient. At step 605, method 600 may comprise obtaining, by the vehicle application 135, medical device data 138 from a plurality of different medical devices 126 deployed in the emergency vehicle 103. At step 607, method 600 may comprise identifying, by the vehicle application 135, using the predictive model 118, a healthcare facility 106 of the plurality of healthcare facilities 106 for treatment of the physician based on the patient data 159, facility data 328, and route traffic data 161.

At step 609, method 600 may comprise determining, by the vehicle application 135, a first network profile 165 for first medical device data 138 received from a first medical device 126 of the different medical devices 126 deployed in the emergency vehicle 103 based on a first policy 168 associated with attributes of at least one of the first medical device 126 or the first medical device data 138. At step 611, method 600 may comprise determining, by the vehicle application 135, a second network profile 165 for second medical device data 138 received from a second medical device 126 of the different medical devices 126 deployed in the emergency vehicle 103 based on a second policy 168 associated with attributes of at least one of the second medical device 126 or the second medical device data 138.

At step 613, method 600 may comprise determining, by the vehicle application 135, a first network path 175 in the communication network 100 along which to route the first medical device data 138 to the healthcare facility 106 based on the first network profile 165 and a second network path 175 in the communication network 100 along which to route the second medical device data 138 based on the second network profile 165. At step 615, method 600 may further comprise instructing, by the vehicle application 135, a network element 129 in the emergency vehicle system 109 to forward the first medical device data 138 to the healthcare facility 106 along the first network path 175 and the second medical device data along the second network path 175.

Method 600 may include additional steps, operations, and elements not explicitly shown in FIG. 6. In an embodiment, the medical device data 138 comprises at least one of videoconferencing data associated with videoconferences occurring in the emergency vehicle 103, real-time vital sign monitoring data, ongoing assessment findings, medical procedure data, patient condition data, or image data associated with images received from cameras deployed in the emergency vehicle 103. In an embodiment, the facility data 328 comprises at least one of a location data 146 indicating a location of the healthcare facility or facility resource data 148 describing an available capacity of resources at the healthcare facility 106. The route traffic data 161 indicates at least one of real-time road conditions, traffic congestions, or potential obstacles encountered along a route to the healthcare facility 106.

In an embodiment, the first network profile 165 is associated with a first network slice 172, wherein the first network path 175 includes resources within the first network slice 172, wherein the second network profile 165 is associated with a second network slice 172, and wherein the second network path 175 includes resources within the second network slice 172. In an embodiment, the different medical devices 126 comprise at least one of a camera, a sensor, a cardiac monitor, a defibrillator, an oxygen delivery system, a suction unit, airway management equipment, a splinting and immobilization device, first aid supplies, intravenous supplies, or diagnostic equipment.

In an embodiment, method 600 may further comprise generating, by the vehicle application 135, using the predictive model 118, a predicted treatment plan for the patient to be performed on the patient while the emergency vehicle is in transit to the healthcare facility, generating, by the vehicle application, using the predictive model, a recommendation for reserving relevant facility resources at the healthcare facility 106 based on the facility resource data 148, and/or generating, by the vehicle application, using the predictive model, a recommendation to initiate contact with a physician of the patient based on the physician data 215, the recommendation including contact information for the physician. In an embodiment, method 600 may further comprise displaying, by the vehicle application 135, a patient summary 303 associated with the patient at a display 132 of the emergency vehicle system 109, wherein the patient summary 303 is a concise overview of a medical history of the patient and relevant health information of the patient.

Turning now to FIG. 7A, an exemplary communication system 550 is described. In an embodiment, the communication system 550 may be implemented in the system 100 of FIG. 1. The communication system 550 includes a number of access nodes 554 that are configured to provide coverage in which UEs 552, such as cell phones, tablet computers, machine-type-communication devices, tracking devices, embedded wireless modules, and/or other wirelessly equipped communication devices (whether or not user operated), or devices such as the medical device 126. The access nodes 554 may be said to establish an access network 556. The access network 556 may be referred to as RAN in some contexts. In a 5G technology generation an access node 554 may be referred to as a gigabit Node B (gNB). In 4G technology (e.g., LTE technology) an access node 554 may be referred to as an eNB. In 3G technology (e.g., CDMA and GSM) an access node 554 may be referred to as a base transceiver station (BTS) combined with a base station controller (BSC). In some contexts, the access node 554 may be referred to as a cell site or a cell tower. In some implementations, a picocell may provide some of the functionality of an access node 554, albeit with a constrained coverage area. Each of these different embodiments of an access node 554 may be considered to provide roughly similar functions in the different technology generations.

In an embodiment, the access network 556 comprises a first access node 554a, a second access node 554b, and a third access node 554c. It is understood that the access network 556 may include any number of access nodes 554. Further, each access node 554 could be coupled with a core network 558 that provides connectivity with various application servers 559 and/or a network 560. In an embodiment, at least some of the application servers 559 may be located close to the network edge (e.g., geographically close to the UE 552 and the end user) to deliver so-called “edge computing.” The network 560 may be one or more private networks, one or more public networks, or a combination thereof. The network 560 may comprise the public switched telephone network (PSTN). The network 560 may comprise the Internet. With this arrangement, a UE 552 within coverage of the access network 556 could engage in air-interface communication with an access node 554 and could thereby communicate via the access node 554 with various application servers and other entities.

The communication system 550 could operate in accordance with a particular radio access technology (RAT), with communications from an access node 554 to UEs 552 defining a downlink or forward link and communications from the UEs 552 to the access node 554 defining an uplink or reverse link. Over the years, the industry has developed various generations of RATs, in a continuous effort to increase available data rate and quality of service for end users. These generations have ranged from “1G,” which used simple analog frequency modulation to facilitate basic voice-call service, to “4G”—such as Long Term Evolution (LTE), which now facilitates mobile broadband service using technologies such as orthogonal frequency division multiplexing (OFDM) and multiple input multiple output (MIMO).

Recently, the industry has been exploring developments in “5G” and particularly “5G NR” (5G New Radio), which may use a scalable OFDM air interface, advanced channel coding, massive MIMO, beamforming, mobile mmWave (e.g., frequency bands above 24 GHz), and/or other features, to support higher data rates and countless applications, such as mission-critical services, enhanced mobile broadband, and massive Internet of Things (IoT). 5G is hoped to provide virtually unlimited bandwidth on demand, for example providing access on demand to as much as 20 gigabits per second (Gbps) downlink data throughput and as much as 10 Gbps uplink data throughput. Due to the increased bandwidth associated with 5G, it is expected that the new networks will serve, in addition to conventional cell phones, general internet service providers for laptops and desktop computers, competing with existing ISPs such as cable internet, and also will make possible new applications in internet of things (IoT) and machine to machine areas.

In accordance with the RAT, each access node 554 could provide service on one or more radio-frequency (RF) carriers, each of which could be frequency division duplex (FDD), with separate frequency channels for downlink and uplink communication, or time division duplex (TDD), with a single frequency channel multiplexed over time between downlink and uplink use. Each such frequency channel could be defined as a specific range of frequency (e.g., in radio-frequency (RF) spectrum) having a bandwidth and a center frequency and thus extending from a low-end frequency to a high-end frequency. Further, on the downlink and uplink channels, the coverage of each access node 554 could define an air interface configured in a specific manner to define physical resources for carrying information wirelessly between the access node 554 and UEs 552.

Without limitation, for instance, the air interface could be divided over time into frames, subframes, and symbol time segments, and over frequency into subcarriers that could be modulated to carry data. The example air interface could thus define an array of time-frequency resource elements each being at a respective symbol time segment and subcarrier, and the subcarrier of each resource element could be modulated to carry data. Further, in each subframe or other transmission time interval (TTI), the resource elements on the downlink and uplink could be grouped to define physical resource blocks (PRBs) that the access node could allocate as needed to carry data between the access node and served UEs 552.

In addition, certain resource elements on the example air interface could be reserved for special purposes. For instance, on the downlink, certain resource elements could be reserved to carry synchronization signals that UEs 552 could detect as an indication of the presence of coverage and to establish frame timing, other resource elements could be reserved to carry a reference signal that UEs 552 could measure in order to determine coverage strength, and still other resource elements could be reserved to carry other control signaling such as PRB-scheduling directives and acknowledgement messaging from the access node 554 to served UEs 552. And on the uplink, certain resource elements could be reserved to carry random access signaling from UEs 552 to the access node 554, and other resource elements could be reserved to carry other control signaling such as PRB-scheduling requests and acknowledgement signaling from UEs 552 to the access node 554.

The access node 554, in some instances, may be split functionally into a radio unit (RU), a distributed unit (DU), and a central unit (CU) where each of the RU, DU, and CU have distinctive roles to play in the access network 556. The RU provides radio functions. The DU provides L1 and L2 real-time scheduling functions; and the CU provides higher L2 and L3 non-real time scheduling. This split supports flexibility in deploying the DU and CU. The CU may be hosted in a regional cloud data center. The DU may be co-located with the RU, or the DU may be hosted in an edge cloud data center.

Turning now to FIG. 7B, further details of the core network 558 are described. In an embodiment, the core network 558 is a 5G core network. 5G core network technology is based on a service based architecture paradigm. Rather than constructing the 5G core network as a series of special purpose communication nodes (e.g., an HSS node, an MME node, etc.) running on dedicated server computers, the 5G core network is provided as a set of services or network functions. These services or network functions can be executed on virtual servers in a cloud computing environment which supports dynamic scaling and avoidance of long-term capital expenditures (fees for use may substitute for capital expenditures). These network functions can include, for example, a user plane function (UPF) 579, an authentication server function (AUSF) 575, an access and mobility management function (AMF) 576, a session management function (SMF) 577, a network exposure function (NEF) 570, a network repository function (NRF) 571, a policy control function (PCF) 572, a unified data management (UDM) 573, a network slice selection function (NSSF) 574, and other network functions. The network functions may be referred to as virtual network functions (VNFs) in some contexts.

Network functions may be formed by a combination of small pieces of software called microservices. Some microservices can be re-used in composing different network functions, thereby leveraging the utility of such microservices. Network functions may offer services to other network functions by extending application programming interfaces (APIs) to those other network functions that call their services via the APIs. The 5G core network 558 may be segregated into a user plane 580 and a control plane 582, thereby promoting independent scalability, evolution, and flexible deployment.

The UPF 579 delivers packet processing and links the UE 552, via the access network 556, to a data network 590 (e.g., the network 560 illustrated in FIG. 7A). The AMF 576 handles registration and connection management of non-access stratum (NAS) signaling with the UE 552. Said in other words, the AMF 576 manages UE registration and mobility issues. The AMF 576 manages reachability of the UEs 552 as well as various security issues. The SMF 577 handles session management issues. Specifically, the SMF 577 creates, updates, and removes (destroys) protocol data unit (PDU) sessions and manages the session context within the UPF 579. The SMF 577 decouples other control plane functions from user plane functions by performing dynamic host configuration protocol (DHCP) functions and IP address management functions. The AUSF 575 facilitates security processes.

The NEF 570 securely exposes the services and capabilities provided by network functions. The NRF 571 supports service registration by network functions and discovery of network functions by other network functions. The PCF 572 supports policy control decisions and flow based charging control. The UDM 573 manages network user data and can be paired with a user data repository (UDR) that stores user data such as customer profile information, customer authentication number, and encryption keys for the information. An application function 592, which may be located outside of the core network 558, exposes the application layer for interacting with the core network 558. In an embodiment, the application function 592 may be executed on an application server 559 located geographically proximate to the UE 552 in an “edge computing” deployment mode. The core network 558 can provide a network slice to a subscriber, for example an enterprise customer, that is composed of a plurality of 5G network functions that are configured to provide customized communication service for that subscriber, for example to provide communication service in accordance with communication policies defined by the customer. The NSSF 574 can help the AMF 576 to select the network slice instance (NSI) for use with the UE 552.

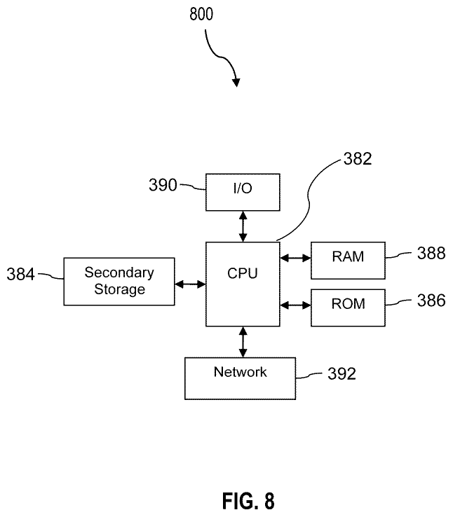

FIG. 8 illustrates a computer system 800 suitable for implementing one or more embodiments disclosed herein. In an embodiment, the emergency vehicle system 109, the healthcare facility systems 112, etc., may each be implemented as the computer system 800. The computer system 800 includes a processor 382 (which may be referred to as a central processor unit or CPU) that is in communication with memory devices including secondary storage 384, read only memory (ROM) 386, random access memory (RAM) 388, input/output (I/O) devices 390, and network connectivity devices 392. The processor 382 may be implemented as one or more CPU chips.

It is understood that by programming and/or loading executable instructions onto the computer system 800, at least one of the CPU 382, the RAM 388, and the ROM 386 are changed, transforming the computer system 800 in part into a particular machine or apparatus having the novel functionality taught by the present disclosure. It is fundamental to the electrical engineering and software engineering arts that functionality that can be implemented by loading executable software into a computer can be converted to a hardware implementation by well-known design rules. Decisions between implementing a concept in software versus hardware typically hinge on considerations of stability of the design and numbers of units to be produced rather than any issues involved in translating from the software domain to the hardware domain. Generally, a design that is still subject to frequent change may be preferred to be implemented in software, because re-spinning a hardware implementation is more expensive than re-spinning a software design. Generally, a design that is stable that will be produced in large volume may be preferred to be implemented in hardware, for example in an application specific integrated circuit (ASIC), because for large production runs the hardware implementation may be less expensive than the software implementation. Often a design may be developed and tested in a software form and later transformed, by well-known design rules, to an equivalent hardware implementation in an application specific integrated circuit that hardwires the instructions of the software. In the same manner as a machine controlled by a new ASIC is a particular machine or apparatus, likewise a computer that has been programmed and/or loaded with executable instructions may be viewed as a particular machine or apparatus.

Additionally, after the system 800 is turned on or booted, the CPU 382 may execute a computer program or application. For example, the CPU 382 may execute software or firmware stored in the ROM 386 or stored in the RAM 388. In some cases, on boot and/or when the application is initiated, the CPU 382 may copy the application or portions of the application from the secondary storage 384 to the RAM 388 or to memory space within the CPU 382 itself, and the CPU 382 may then execute instructions that the application is comprised of. In some cases, the CPU 382 may copy the application or portions of the application from memory accessed via the network connectivity devices 392 or via the I/O devices 390 to the RAM 388 or to memory space within the CPU 382, and the CPU 382 may then execute instructions that the application is comprised of. During execution, an application may load instructions into the CPU 382, for example load some of the instructions of the application into a cache of the CPU 382. In some contexts, an application that is executed may be said to configure the CPU 382 to do something, e.g., to configure the CPU 382 to perform the function or functions promoted by the subject application. When the CPU 382 is configured in this way by the application, the CPU 382 becomes a specific purpose computer or a specific purpose machine.

The secondary storage 384 is typically comprised of one or more disk drives or tape drives and is used for non-volatile storage of data and as an over-flow data storage device if RAM 388 is not large enough to hold all working data. Secondary storage 384 may be used to store programs which are loaded into RAM 388 when such programs are selected for execution. The ROM 386 is used to store instructions and perhaps data which are read during program execution. ROM 386 is a non-volatile memory device which typically has a small memory capacity relative to the larger memory capacity of secondary storage 384. The RAM 388 is used to store volatile data and perhaps to store instructions. Access to both ROM 386 and RAM 388 is typically faster than to secondary storage 384. The secondary storage 384, the RAM 388, and/or the ROM 386 may be referred to in some contexts as computer readable storage media and/or non-transitory computer readable media.

I/O devices 390 may include printers, video monitors, liquid crystal displays (LCDs), touch screen displays, keyboards, keypads, switches, dials, mice, track balls, voice recognizers, card readers, paper tape readers, or other well-known input devices.

The network connectivity devices 392 may take the form of modems, modem banks, Ethernet cards, universal serial bus (USB) interface cards, serial interfaces, token ring cards, fiber distributed data interface (FDDI) cards, wireless local area network (WLAN) cards, radio transceiver cards, and/or other well-known network devices. The network connectivity devices 392 may provide wired communication links and/or wireless communication links (e.g., a first network connectivity device 392 may provide a wired communication link and a second network connectivity device 392 may provide a wireless communication link). Wired communication links may be provided in accordance with Ethernet (IEEE 802.3), Internet protocol (IP), time division multiplex (TDM), data over cable service interface specification (DOCSIS), wavelength division multiplexing (WDM), and/or the like. In an embodiment, the radio transceiver cards may provide wireless communication links using protocols such as code division multiple access (CDMA), global system for mobile communications (GSM), long-term evolution (LTE), WiFi (IEEE 802.11), Bluetooth, Zigbee, narrowband Internet of things (NB IoT), near field communications (NFC), and radio frequency identity (RFID). The radio transceiver cards may promote radio communications using 5G, 5G New Radio, or 5G LTE radio communication protocols. These network connectivity devices 392 may enable the processor 382 to communicate with the Internet or one or more intranets. With such a network connection, it is contemplated that the processor 382 might receive information from the network, or might output information to the network in the course of performing the above-described method steps. Such information, which is often represented as a sequence of instructions to be executed using processor 382, may be received from and outputted to the network, for example, in the form of a computer data signal embodied in a carrier wave.

Such information, which may include data or instructions to be executed using processor 382 for example, may be received from and outputted to the network, for example, in the form of a computer data baseband signal or signal embodied in a carrier wave. The baseband signal or signal embedded in the carrier wave, or other types of signals currently used or hereafter developed, may be generated according to several methods well-known to one skilled in the art. The baseband signal and/or signal embedded in the carrier wave may be referred to in some contexts as a transitory signal.

The processor 382 executes instructions, codes, computer programs, scripts which it accesses from hard disk, floppy disk, optical disk (these various disk based systems may all be considered secondary storage 384), flash drive, ROM 386, RAM 388, or the network connectivity devices 392. While only one processor 382 is shown, multiple processors may be present. Thus, while instructions may be discussed as executed by a processor, the instructions may be executed simultaneously, serially, or otherwise executed by one or multiple processors. Instructions, codes, computer programs, scripts, and/or data that may be accessed from the secondary storage 384, for example, hard drives, floppy disks, optical disks, and/or other device, the ROM 386, and/or the RAM 388 may be referred to in some contexts as non-transitory instructions and/or non-transitory information.

In an embodiment, the computer system 800 may comprise two or more computers in communication with each other that collaborate to perform a task. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers. In an embodiment, virtualization software may be employed by the computer system 800 to provide the functionality of a number of servers that is not directly bound to the number of computers in the computer system 800. For example, virtualization software may provide twenty virtual servers on four physical computers. In an embodiment, the functionality disclosed above may be provided by executing the application and/or applications in a cloud computing environment. Cloud computing may comprise providing computing services via a network connection using dynamically scalable computing resources. Cloud computing may be supported, at least in part, by virtualization software. A cloud computing environment may be established by an enterprise and/or may be hired on an as-needed basis from a third-party provider. Some cloud computing environments may comprise cloud computing resources owned and operated by the enterprise as well as cloud computing resources hired and/or leased from a third-party provider.

In an embodiment, some or all of the functionality disclosed above may be provided as a computer program product. The computer program product may comprise one or more computer readable storage medium having computer usable program code embodied therein to implement the functionality disclosed above. The computer program product may comprise data structures, executable instructions, and other computer usable program code. The computer program product may be embodied in removable computer storage media and/or non-removable computer storage media. The removable computer readable storage medium may comprise, without limitation, a paper tape, a magnetic tape, magnetic disk, an optical disk, a solid state memory chip, for example analog magnetic tape, compact disk read only memory (CD-ROM) disks, floppy disks, jump drives, digital cards, multimedia cards, and others. The computer program product may be suitable for loading, by the computer system 800, at least portions of the contents of the computer program product to the secondary storage 384, to the ROM 386, to the RAM 388, and/or to other non-volatile memory and volatile memory of the computer system 800. The processor 382 may process the executable instructions and/or data structures in part by directly accessing the computer program product, for example by reading from a CD-ROM disk inserted into a disk drive peripheral of the computer system 800. Alternatively, the processor 382 may process the executable instructions and/or data structures by remotely accessing the computer program product, for example by downloading the executable instructions and/or data structures from a remote server through the network connectivity devices 392. The computer program product may comprise instructions that promote the loading and/or copying of data, data structures, files, and/or executable instructions to the secondary storage 384, to the ROM 386, to the RAM 388, and/or to other non-volatile memory and volatile memory of the computer system 800.

In some contexts, the secondary storage 384, the ROM 386, and the RAM 388 may be referred to as a non-transitory computer readable medium or a computer readable storage media. A dynamic RAM embodiment of the RAM 388, likewise, may be referred to as a non-transitory computer readable medium in that while the dynamic RAM receives electrical power and is operated in accordance with its design, for example during a period of time during which the computer system 800 is turned on and operational, the dynamic RAM stores information that is written to it. Similarly, the processor 382 may comprise an internal RAM, an internal ROM, a cache memory, and/or other internal non-transitory storage blocks, sections, or components that may be referred to in some contexts as non-transitory computer readable media or computer readable storage media.

While several embodiments have been provided in the present disclosure, it should be understood that the disclosed systems and methods may be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted or not implemented.

Also, techniques, systems, subsystems, and methods described and illustrated in the various embodiments as discrete or separate may be combined or integrated with other systems, modules, techniques, or methods without departing from the scope of the present disclosure. Other items shown or discussed as directly coupled or communicating with each other may be indirectly coupled or communicating through some interface, device, or intermediate component, whether electrically, mechanically, or otherwise. Other examples of changes, substitutions, and alterations are ascertainable by one skilled in the art and could be made without departing from the spirit and scope disclosed herein.

What is claimed is:

1. A method implemented in a communication network to optimize emergency vehicle to hospital transportation and communications, wherein the method comprises:
obtaining, by a vehicle application implemented by an emergency vehicle system in the communication network, patient data associated with a patient based on identification data of the patient, wherein the patient data comprises historical patient data describing at least one of a diagnosis history of the patient, a treatment history of the patient, physician data indicating contact information of one or more current physicians of the patient, or allergy data identifying one or more allergies of the patient;
obtaining, by the vehicle application, medical device data from a plurality of different medical devices deployed in the emergency vehicle;
identifying, by the vehicle application, using a predictive model in the communications network, a healthcare facility of a plurality of healthcare facilities for treatment of the patient based on the patient data, facility data, and route traffic data;
determining, by the vehicle application, a first network profile for first medical device data received from a first medical device of the different medical devices deployed in the emergency vehicle based on a first policy associated with attributes of at least one of the first medical device or the first medical device data;
determining, by the vehicle application, a second network profile for second medical device data received from a second medical device of the different medical devices deployed in the emergency vehicle based on a second policy associated with attributes of at least one of the second medical device or the second medical device data;
determining, by the vehicle application, a first network path in the communication network along which to route the first medical device data to the healthcare facility based on the first network profile and a second network path in the communication network along which to route the second medical device data based on the second network profile; and
instructing, by the vehicle application, a network element in the emergency vehicle to forward the first medical device data to the healthcare facility along the first network path and the second medical device data along the second network path.

2. The method of claim 1, wherein the medical device data comprises at least one of videoconferencing data associated with videoconferences occurring in the emergency vehicle, real-time vital sign monitoring data, ongoing assessment findings, medical procedure data, patient condition data, or image data associated with images received from cameras deployed in the emergency vehicle.

3. The method of claim 1, wherein the facility data comprises at least one of a location data describing a location of the healthcare facility or facility resource data describing an available capacity of resources at the healthcare facility, and wherein the route traffic data indicates at least one of real-time road conditions, traffic congestions, or potential obstacles encountered along a route to the healthcare facility.

4. The method of claim 1, wherein the first network profile is associated with a first network slice, wherein the first network path includes resources within the first network slice, wherein the second network profile is associated with a second network slice, and wherein the second network path includes resources within the second network slice.

5. The method of claim 1, wherein the different medical devices comprise at least one of a camera, a sensor, a cardiac monitor, a defibrillator, an oxygen delivery system, a suction unit, airway management equipment, a splinting and immobilization device, first aid supplies, intravenous supplies, or diagnostic equipment.

6. The method of claim 1, wherein, while the emergency vehicle is in transit to the healthcare facility, the method further comprises at least one of:
generating, by the vehicle application, using the predictive model, a predicted treatment plan for the patient to be performed on the patient;
initiating, by the vehicle application, using the predictive model, a recommendation to initiate contact with a physician of the patient based on the physician data, wherein the recommendation comprises contact information for the physician; or
generating, by the vehicle application, using the predictive model, a recommendation for reserving relevant facility resources at the healthcare facility based on the facility data.

7. The method of claim 1, further comprising displaying, by the vehicle application, a patient summary associated with the patient at a display of the emergency vehicle system, wherein the patient summary is a concise overview of a medical history of the patient and relevant health information of the patient.

8. A method implemented in a communication network to optimize emergency vehicle to healthcare facility transportation and communications, wherein the method comprises:
obtaining, by a vehicle application implemented by an emergency vehicle system in the communication network, patient data associated with a patient based on identification data of the patient, wherein the patient data comprises historical patient data describing at least one of a diagnosis history of the patient, a treatment history of the patient, physician data indicating contact information of one or more current physicians of the patient, or allergy data identifying one or more allergies of the patient;
obtaining, by the vehicle application, medical device data from a plurality of different medical devices deployed in the emergency vehicle;
obtaining, by the vehicle application, current patient condition data indicating a current condition of the patient based on at least one of the medical device data collected from the different medical devices or input received at the emergency vehicle system;
identifying, by the vehicle application, using a predictive model in the communications network, a healthcare facility for treatment of the patient based on the patient data, the current patient condition data, facility data, and route traffic data, wherein the facility data comprises at least one of a location data indicating a location of the healthcare facility or facility resource data describing an available capacity of resources at the healthcare facility, and wherein the route traffic data indicates at least one of real-time road conditions, traffic congestions, or potential obstacles encountered along a route to the healthcare facility;
generating, by the vehicle application, using the predictive model, a predicted treatment plan for the patient to be performed on the patient while the emergency vehicle is in transit to the healthcare facility; and
instructing, by the vehicle application, a network element to transmit first medical device data from a first medical device along a network path in the communication network to the healthcare facility based on a network profile associated with the first medical device data.

9. The method of claim 8, wherein when the current patient condition data indicates that the patient is in a critical condition and in need of immediate medical care, the identifying the healthcare facility for treatment of the patient is further based on a distance between a current location of the patient and the location of the healthcare facility.

10. The method of claim 8, wherein when the current patient condition data indicates that the patient needs surgery to treat an injury, the identifying the healthcare facility for treatment of the patient is further based on a surgeons and surgical resources available at the healthcare facility.

11. The method of claim 8, wherein while the emergency vehicle is in transit to the healthcare facility, the method further comprises initiating, by the vehicle application, using the predictive model, a recommendation to initiate contact with a physician of the patient based on the physician data, wherein the recommendation comprises contact information for the physician.

12. The method of claim 8, wherein while the emergency vehicle is in transit to the healthcare facility, the method further comprises generating, by the vehicle application, using the predictive model, a recommendation for reserving relevant facility resources at the healthcare facility based on the facility resource data.

13. The method of claim 8, wherein the medical device data comprises at least one of videoconferencing data associated with videoconferences occurring in the emergency vehicle, real-time vital sign monitoring data, ongoing assessment findings, medical procedure data, patient condition data, or image data associated with images received from cameras deployed in the emergency vehicle.

14. The method of claim 8, further comprising:
obtaining, by the vehicle application, using the predictive model, a patient summary associated with the patient based on the patient data, wherein the patient summary is a concise overview of a medical history of the patient and relevant health information of the patient; and
displaying, by the vehicle application, the patient summary at a display of the emergency vehicle system.

15. A vehicle system of a vehicle, comprising:
at least one processor;
at least one memory coupled to the processor; and
a vehicle application, stored in the at least one memory, which when executed by the at least one processor, causes the vehicle application to be configured to:
obtain patient data associated with a patient based on identification data of the patient, wherein the patient data comprises historical patient data describing at least one of a diagnosis history of the patient, a treatment history of the patient, physician data indicating contact information of one or more current physicians of the patient, or allergy data identifying one or more allergies of the patient;
obtain medical device data from a plurality of different medical devices deployed in the vehicle;
determine current patient condition data indicating a current condition of the patient based on at least one of the medical device data collected from the different medical devices or input received at the vehicle system;
identify using a predictive model in the communications network, a healthcare facility of a plurality of healthcare facilities for treatment of the patient based on the patient data, facility data, and route traffic data; and
instruct a network element to transmit first medical device data from a first medical device along a network path in the communication network along to the healthcare facility based on a network profile associated with the first medical device data.

16. The vehicle system of claim 15, wherein the medical device data comprises at least one of videoconferencing data associated with videoconferences occurring in the vehicle, real-time vital sign monitoring data, ongoing assessment findings, medical procedure data, patient condition data, or image data associated with images received from cameras deployed in the vehicle.

17. The vehicle system of claim 15, wherein the facility data comprises at least one of a location data describing a location of the healthcare facility or facility resource data describing an available capacity of resources at the healthcare facility, and wherein the route traffic data indicates at least one of real-time road conditions, traffic congestions, or potential obstacles encountered along a route to the healthcare facility.

18. The vehicle system of claim 15, wherein the instructions further cause the vehicle application to be configured to:
obtain using the predictive model, a patient summary associated with the patient based on the patient data, wherein the patient summary is a concise overview of a medical history of the patient and relevant health information of the patient; and
display the patient summary at a display of the vehicle system.

19. The vehicle system of claim 15, wherein the instructions further cause the vehicle application to be configured to initiate, using the predictive model, a recommendation to initiate contact with a physician of the patient based on the physician data while the vehicle is in transit to the healthcare facility, wherein the recommendation comprises contact information for the physician.

20. The vehicle system of claim 15, wherein the instructions further cause the vehicle application to be configured to generate, using the predictive model, a recommendation for reserving relevant facility resources at the healthcare facility based on the facility data while the vehicle is in transit to the healthcare facility.
